(19) United States
(12) Patent Application Publication
(10) Pub. No.: US 2021/0275546 A1
(43) Pub. Date: Sep. 9, 2021

(54) HIGH DRUG LOAD SOLID ORAL DOSAGE FORMS OF DEXAMETHASONE

(71) Applicant: Jubilant Pharma Holdings Inc., Yardley, PA (US)

(72) Inventor: Indranil Nandi, Yardley, PA (US)

(73) Assignee: Jubilant Pharma Holdings Inc., Yardley, PA (US)

(21) Appl. No.: 17/319,487

(22) Filed: May 13, 2021

Related U.S. Application Data
(60) Provisional application No. 63/025,001, filed on May 14, 2020.

(51) Int. Cl. A61K 31/573; A61K 9/00; A61K 9/20

(52) U.S. Cl. CPC A61K 31/573; A61K 9/0053; A61K 9/2018; A61K 9/2009; A61K 9/2059; A61K 9/2013; A61K 9/2027; A61K 9/2054

(57) ABSTRACT

The present invention relates to high drug load solid oral pharmaceutical compositions comprising dexamethasone or its pharmaceutically acceptable salts or solvates thereof. The present invention also relates to a process for preparing high drug load solid oral pharmaceutical compositions comprising dexamethasone or its pharmaceutically acceptable salts or solvates thereof. The preferred drug load in the compositions of the present invention is from about 26% to 50% by weight based on the total weight of the composition. The prepared compositions of dexamethasone as per the present invention exhibit desirable technical attributes.

### FIELD OF THE INVENTION

The present invention relates to solid oral pharmaceutical compositions of dexamethasone or its pharmaceutically acceptable salts or solvates. In particular, but without restriction to the particular embodiments hereinafter described in accordance with the best mode of practice, the present invention provides a high drug load solid oral pharmaceutical composition comprising dexamethasone or its pharmaceutically acceptable salts or solvates and processes for preparing the same.

### BACKGROUND OF THE INVENTION

Dexamethasone is a synthetic steroidal glucocorticoid, which is used in the treatment of many conditions, including various skin diseases, severe allergies, asthma, chronic obstructive lung disease, rheumatic problems, and along with antibiotics in tuberculosis, to name a few. Dexamethasone is chemically known as 9-fluoro-11β,17,21trihydroxy-16α-methylpregna-1,4-diene-3,20-dione. Dexamethasone is practically insoluble in water, sparingly soluble in acetone, ethanol, and methanol, and slightly soluble in dichloromethane and is represented by the following formula as:

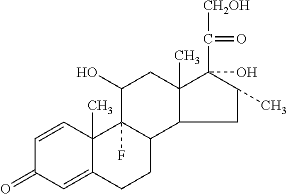

Solid oral dosage forms of dexamethasone are approved in the US market as immediate-release tablets in strengths of 0.25 mg, 0.5 mg, 0.75 mg, 1 mg, 1.5 mg, 2 mg, 4 mg, and 6 mg for the treatment of various diseases such as allergic, dermatologic, endocrine, gastrointestinal, hematologic, renal, respiratory, rheumatic diseases and palliative management of leukemias and lymphomas.

Recently, dexamethasone was also approved as HEMADY® by Dexcel Pharma as an immediate-release tablet in a strength of 20 mg. The HEMADY® tablet contains dexamethasone along with inactive ingredients such as corn starch, lactose monohydrate, magnesium stearate, povidone, and sodium starch glycolate.

Dexamethasone is reported to be poorly water-soluble, which leads to poor dissolution and bioavailability. Poor aqueous solubility, flowability, and compressibility of the drug also pose technical challenges to formulation scientists in the development of a suitable formulation with desired technical attributes.

U.S. Pat. No. 10,537,585 assigned to Dexcel Pharma discloses high dose dexamethasone compositions. The said patent further discloses a pharmaceutical composition comprising dexamethasone in an amount of from about 15 to about 25 weight percent along with other excipients such as a filler, a binder, a disintegrant, and a lubricant.

US Patent Publication No. 2006/0034915 assigned to Constant Research & Development discloses a tablet composition, wherein dexamethasone is present in an amount of from 2% to 0.02% w/w and a viscosity increasing excipient (starch) is present in an amount of from 0.5% to 5% w/w. The said patent publication highlights that dexamethasone is a potent corticosteroid, a small amount of drug is needed in oral dosage form, i.e., 0.25 mg in 300 mg tablet (0.083%). The said patent publication further highlights that it is challenging to uniformly disperse small amounts of the drug in tablet granulation.

The pharmaceutical compositions of dexamethasone suitable for oral administration to humans must have desirable chemical and physical properties, dissolution, stability, and bioequivalence complying with demanding requirements and regulations of health and medicine regulatory agencies across the world.

Thus, there is a need for an alternate dosage form of dexamethasone with desirable technical formulation attributes such as disintegration, dissolution, improved flow characteristics such as bulk density, tapped density, Hausner ratio, compressibility index, content uniformity, stability, and bioequivalence, and which can be manufactured by a simple, reproducible and commercially viable process at lab scale as well as at industrial scale.

The present inventors have developed an alternate dosage form of dexamethasone, which offers desirable formulation characteristics like disintegration, dissolution, improved flow characteristics such as bulk density, tapped density, Hausner ratio, compressibility index, content uniformity, stability, and bioequivalence which is comparable to the commercially available counterpart (HEMADY® Tablets). Further, the process employed in the manufacture of the dosage form of dexamethasone is consistent and therefore feasible for industrial production.

### OBJECTS AND SUMMARY OF THE INVENTION

The present invention relates to high drug load solid oral pharmaceutical compositions comprising dexamethasone or its pharmaceutically acceptable salts or solvates and process for preparing such compositions.

The present invention also relates to high drug load solid oral pharmaceutical compositions comprising dexamethasone or its pharmaceutically acceptable salts or solvates and one or more pharmaceutically acceptable excipients and processes for preparing such compositions.

The present invention also relates to high drug load solid oral pharmaceutical compositions comprising dexamethasone or its pharmaceutically acceptable salts or solvates and one or more pharmaceutically acceptable excipients, like diluent, binder, disintegrant, lubricant, glidant, and surfactant.

The present invention further relates to high drug load solid oral pharmaceutical compositions comprising dexamethasone or its pharmaceutically acceptable salts or solvates and one or more pharmaceutically acceptable excipients, which exhibits desired pharmaceutical technical attributes such as disintegration, dissolution, improved flow characteristics such as bulk density, tapped density, Hausner ratio, compressibility index, content uniformity, stability, and bioequivalence.

The present invention also relates to the use of high drug load solid oral pharmaceutical compositions of the present invention in the manufacture of medicaments for treating multiple myeloma in combination with at least one anti-cancer drug, allergy states, dermatological diseases, endocrine disorders, gastrointestinal diseases, hematological diseases, neoplastic diseases, nervous system disorders, ophthalmic diseases, renal diseases, respiratory diseases, rheumatic diseases, trichinosis, and tuberculous meningitis.

### DETAILED DESCRIPTION

The present invention can be more readily understood by reading the following detailed description of the invention and study of the included examples.

As used herein, the term “composition”, as in pharmaceutical composition, is intended to encompass a drug product comprising dexamethasone or its pharmaceutically acceptable salts, esters, solvates, polymorphs, enantiomers or mixtures thereof, and other inert ingredient(s) (pharmaceutically acceptable excipients). Such pharmaceutical compositions are synonymous with “formulation” and “dosage form”. Pharmaceutical compositions of the invention include, but are not limited to, granules, tablets (single-layered tablets, multilayered tablets, bioadhesive tablets, caplets, matrix tablets, tablet within a tablet, mucoadhesive tablets, immediate-release tablets, modified-release tablets, pulsatile release tablets, timed-release tablets, delayed-release, controlled release, extended-release, and sustained-release tablets), capsules (immediate or modified release) (hard and soft or liquid-filled soft gelatin capsules), pills, troches, sachets, powders, microcapsules, mini-tablets, tablets in capsules and microspheres, matrix composition and the like. Preferably, the pharmaceutical composition refers to tablets and capsules. More preferably, the pharmaceutical composition refers to immediate-release oral tablets, which may be uncoated or film-coated. More preferably, the pharmaceutical composition refers to uncoated immediate-release oral tablets. The tablet dosage forms as per the present invention are capable of withstanding during packaging, transportation, and use without any defect.

The term “immediate release,” as used herein, implies that dexamethasone is released from the composition in an immediate release fashion such that composition releases more than 70% of the dexamethasone in 45 minutes or less. In an embodiment, the composition release more than 70% of the dexamethasone in 45 minutes or less across all physiological pH ranges such as 1.2, 4.5, and 6.8. In an embodiment, the composition exhibits more than 70% of drug release within 45 minutes in 500 ml of Acetate buffer, pH 4.5, using USP II apparatus (Paddle) at a temperature of 37±0.5° C. and a rotation speed of 50 revolutions per minute. In an embodiment, the composition exhibits more than 70% of drug release within 45 minutes in 500 ml of Phosphate buffer, pH 6.8, using USP II apparatus (Paddle) at a temperature of 37±0.5° C. and a rotation speed of 50 revolutions per minute. In an embodiment, the composition exhibits more than 80% of drug release within 30 minutes in 900 ml of dilute Hydrochloric acid, using USP II apparatus (Paddle) at a temperature of 37±0.5° C. and a rotation speed of 50 revolutions per minute. In another embodiment, the composition exhibits more than 70% of drug release within 45 minutes in 900 ml of dilute Hydrochloric acid, using USP I apparatus (Basket) at a temperature of 37±0.5° C. and a rotation speed of 50 revolutions per minute. In an embodiment, the composition releases more than 70% of the dexamethasone in 45 minutes in 500 ml of 0.1N Hydrochloric acid, using apparatus 1 (Basket), at a temperature of 37±0.5° C., and a rotation speed of 50 revolutions per minute or more than 70% of drug within 45 minutes in 500 ml of 0.1N Hydrochloric acid, using USP II apparatus (Paddle) at a temperature of 37±0.5° C. and a rotation speed of 50 revolutions per minute.

As used herein, the term “dexamethasone” is used in a broad sense to include not only “dexamethasone” per se (free base) but also its pharmaceutically acceptable salts, solvates, hydrates, enantiomers, derivatives, isomers, metabolites, polymorphs, prodrugs thereof. Polymorph may refer to various crystalline and amorphous forms of dexamethasone, which can be characterized by methods such as melting point, X-ray diffraction pattern, Raman spectra, IR spectra, or any other method known in the art. Suitable salts of dexamethasone include but are not limited to, acid addition salts and base addition salts such as sodium, potassium, zinc, magnesium, hydrochloride, hydrobromide, sulfate, phosphate, nitrate and like, amino acids salts such as glycine, lysine, arginine, or any other known salt in the art.

The term “high drug load” as used herein, refers to an amount of from about 20% to about 97% by weight of dexamethasone based on the total weight of the composition. Preferably, the high drug load solid oral pharmaceutical composition as per the present invention comprises from about 25.1% to about 50%, about 26% to about 50% of dexamethasone. More preferably, the high drug load solid oral pharmaceutical composition as per the present invention comprises from about 26% to about 35% of dexamethasone. The high drug load solid oral pharmaceutical composition as per the present invention comprises more than 25% of dexamethasone. More preferably, high drug load solid oral pharmaceutical compositions as per the present invention comprise about 27.5% or more than 27.5% of dexamethasone. Preferably, the high drug load solid oral pharmaceutical composition as per the present invention does not comprise less than 20% of dexamethasone.

The term “excipient” means a pharmacologically inactive component such as a diluent, binder, disintegrant, glidant, surfactant, wetting agent, lubricant, solid carrier, solubilizer, stabilizer, sweetener, flavoring agent, coloring agent, and the like. Co-processed excipients are also covered under the scope of the present invention. The excipient(s) may be present intra-granularly or extra-granularly or in both the phases in any form. Further, excipients may be in the form of powders or in the form of a dispersion. Combinations of excipients performing the same function may also be used to achieve desired formulation characteristics.

“Substantially free” as used herein refers to the pharmaceutical composition of dexamethasone, which is devoid of said element/component/excipient or may contain less than 10% or less than 5% or less than 2% or preferably less than 1% of the said element/component/excipient. The term “starch” includes but is not limited to corn starch, pregelatinized starch, sodium carboxymethyl starch, microcrystalline starch, or other starch derivatives thereof. The term “free of starch” as used herein refers to the pharmaceutical composition of dexamethasone, which does not contain any starch excipient.

As used herein, the term “intra-granular” (part/phase/portion) refers to the components of formulation of the present invention that are within granules. As used herein, the term “extra-granular” (part/phase/portion) refers to those components of the formulation of the present invention that are outside the granules.

Unless otherwise stated the weight percentages expressed herein are based on the final weight of the composition or formulation.

As used herein, the term “about” means±approximately 20% of the indicated value, such that “about 10 percent” indicates approximately 8 to 12 percent.

As used herein, the term “therapeutic agent” means an agent utilized to treat, combat, ameliorate, prevent or improve an unwanted condition or allergy, or disease of a patient. “Administering” composition may be accomplished by oral administration and/or by other methods alone or in combination with other known techniques. Preferably, the composition according to the present invention is administered orally.

The compositions prepared by the processes as per the present invention are tested for physical parameters such as weight variation, hardness, thickness, bulk density, tapped density, assay, content uniformity, disintegration test, friability, compressibility index, Hausner ratio, disintegration, and dissolution. Several devices can be used to test tablet hardness such as a Monsanto tester, a Strong-Cobb tester, a Pfizer tester, an Erweka tester, a Schleuniger tester, etc. In some embodiments, the tablet according to the present invention exhibits a target hardness of about 30 N to about 150 N. In an embodiment, the hardness of the tablet according to the present invention is about 150 N or less, e.g., 140 N or less, 130 N or less, 120 N or less, 110 N or less, 100 N or less, 90 N or less, 80 N or less. In some embodiments, the tablet according to the present invention exhibits a target hardness of about 30 N to about 110 N.

Friability can be determined using a Roche friabilator for 100 revolutions at 25 RPM. In some embodiments, the tablet according to the present invention exhibits friability of about 1% or less. In an embodiment, the friability of the tablet according to the present invention is about 1% or less, e.g., 0.9% or less, 0.8% or less, 0.7% or less, 0.6% or less, 0.5% or less, 0.4% or less, 0.3% or less, 0.2% or less. In an embodiment, the friability of the tablet according to the present invention is about 0.5% or less.

Disintegration time testing for tablets can be performed in a USP tablet disintegration tester wherein a tablet is placed in a basket, which moves upward and downward in a 1-liter beaker of water at 37° C. The compositions prepared by the process as per the present invention can be subjected to in vitro dissolution evaluation according to Test 711 “Dissolution” in the United States Pharmacopoeia 37, United States Pharmacopoeial Convention, Inc., Rockville, Md., 2014 (“USP”) to determine the rate at which the active substance is released from the dosage form, and the content of the active substance can be determined in solution by high-performance liquid chromatography. When comparing the test and reference products, dissolution profiles should be compared using a similarity factor (f2). The similarity factor is a logarithmic reciprocal square root transformation of the sum of squared error and is a measurement of the similarity in the percent (%) of dissolution between the two curves. Two dissolution profiles are considered similar when the f2 value is equal to or greater than 50.

The term “stable” refers to the compositions of the present invention in which the amount of active ingredient of a formulation does not deviate from the initial amount by more than the values given in the specification or the guidelines of the common Pharmacopoeias or loss in active ingredient is less than 50% of the initial content after being stored for at least 1 month, preferably for at least 2 months, preferably for at least 3 months, more preferably for at least 6 months, more preferably for at least 12 months or more preferably for at least 24 months. The stability of the high drug load solid oral composition may be evaluated at “long term” conditions 25° C./60% RH, at intermediate condition 30° C./65% RH, at “accelerated conditions” 40° C./75% RH, in the final container either measured as the loss in the content of active ingredient. Stability testing may be conducted according to the current guidelines by ICH (International Council for Harmonisation) and USFDA (United States Food and Drug Administration).

The pharmaceutical compositions of the present invention comprise about 0.1 to about 50 mg of dexamethasone, preferably about 2 to about 40 mg of dexamethasone, more preferably about 20 mg of dexamethasone. The pharmaceutical composition comprises dexamethasone in the range of about 20% to about 97% by weight, preferably in the range of about 26% to about 50% by weight, more preferably in the range of about 27.5% to about 35% by weight on the basis of the total weight of the composition. In another embodiment according to the present invention, the high drug load solid oral pharmaceutical composition of the present invention includes dexamethasone as the sole active ingredient. In another embodiment, the composition comprises 20 mg of dexamethasone.

In another embodiment the high drug load pharmaceutical composition of the present invention includes particle sizes of dexamethasone or its pharmaceutically acceptable salts or solvates, having a particle size distribution such that D₉₀ is less than about 200 μm, D₅₀ is less than about 100 μm and D₁₀ is less than about 50 μm. Preferably, the particle size distribution is D₉₀ is less than about 100 μm, D₅₀ is less than about 70 μm and D₁₀ is less than about 30 μm. The particle size of dexamethasone can be measured by suitable techniques such as Laser light scattering (e.g. Malvern Light Scattering), Coulter counter, microscopy, and any other technique known in the art. In an embodiment, the D₉₀ according to the present invention is present in about 200 μm or less, e.g., 180 μm or less, 140 μm or less, 100 μm or less, 80 μm or less, 60 μm or less, 40 μm or less, 20 μm or less, 10 μm or less. In an embodiment, the D₅₀ according to the present invention is present in about 100 μm or less, e.g., 80 μm or less, 60 μm or less, 50 μm or less, 40 μm or less, 20 μm or less, 10 μm or less. In an embodiment, the D₁₀ according to the present invention is present is about 50 μm or less, e.g., 40 μm or less, 30 μm or less, 20 μm or less, 10 μm or less, 5 μm or less.

In another embodiment of the invention, the high drug load solid oral pharmaceutical composition comprising dexamethasone is prepared by a wet or dry process. The wet and dry processes include, but are not limited to, wet granulation, dry granulation, dry blending, dry mixing, and direct compression. Other formulation techniques are also contemplated within the scope of the present invention. Any pharmaceutically acceptable granulating agent can be used for wet granulation. Preferably, granulating solvents include, but are not limited to, water, esters such as ethyl acetate, ketones such as acetone, alcohols such as methanol, ethanol, isopropanol, butanol, dichloromethane, chloroform, dimethylacetamide (DMA), dimethyl sulfoxide (DMSO), ether, diethyl ether and combinations thereof. Preferably, the granulating solvent used during wet granulation is water. In another embodiment of the invention, wet granulation can be performed using a rapid mixer granulator, a fluid bed granulator, a planetary mixer, and the like; dry blending can be performed using V-blender or key blender, and dry granulation can be performed using a roller compacter or slugging techniques or by any other method known in the art. The granules obtained as per the present invention are dried at a suitable temperature in a suitable drying apparatus until the desired loss on drying value is obtained. In an embodiment, the Loss On Drying (LOD) of the granulate is in the range of about 1% to about 7%, preferably from about 1% to about 5%. The water content of the tablets can be measured using the loss on drying method or the Karl Fischer method, which are well known to those skilled in the art.

The present invention relates to high drug load solid oral pharmaceutical composition comprising dexamethasone or its pharmaceutically acceptable salts or solvates thereof and processes for preparing such compositions.

In another embodiment of the invention, there is provided a high drug load solid oral pharmaceutical composition comprising dexamethasone or its pharmaceutically acceptable salts or solvates thereof and one or more pharmaceutically acceptable excipients and process for preparing such compositions.

In another embodiment of the invention, there is provided a high drug load solid oral pharmaceutical composition comprising dexamethasone or its pharmaceutically acceptable salts or solvates thereof and one or more pharmaceutically acceptable excipients, like diluent, binder, disintegrant, surfactant, glidant, and lubricant.

In another embodiment of the invention, there is provided a high drug load solid oral pharmaceutical composition comprising dexamethasone or its pharmaceutically acceptable salts or solvates thereof in an amount of from about 25.1% to about 50% by weight and one or more pharmaceutically acceptable excipients, like diluent, binder, disintegrant, surfactant, glidant, and lubricant.

In another embodiment of the invention, there is provided a high drug load solid oral pharmaceutical composition comprising dexamethasone or its pharmaceutically acceptable salts or solvates thereof in an amount of from about 26% to about 50% by weight and one or more pharmaceutically acceptable excipients, like diluent, binder, disintegrant, surfactant, glidant, and lubricant.

In another embodiment of the invention, there is provided a high drug load solid oral pharmaceutical composition comprising dexamethasone or its pharmaceutically acceptable salts or solvates thereof present in an amount of more than 25% by weight based on the total weight of the composition. In particular, the amount of dexamethasone or its pharmaceutically acceptable salts or solvates thereof may vary from about 26% to about 50% by weight, preferably, from about 27.5% to about 35% by weight, based on the total weight of the composition.

In another embodiment of the invention, there is provided a high drug load solid oral pharmaceutical composition of dexamethasone or its pharmaceutically acceptable salts or solvates thereof that make the granulation process more consistent and therefore feasible for industrial production, while maintaining stability and pharmaceutical equivalence to the reference listed drug.

In another embodiment, the present invention includes a high drug load solid oral pharmaceutical composition of dexamethasone comprising from about 26% to about 50% by weight of dexamethasone, preferably from about 27.5% to about 35% by weight of dexamethasone based on the total weight of the composition wherein, the composition is substantially free of any starch excipient. In a further embodiment, the composition as per the present invention is devoid of any starch excipient.

In another embodiment, the present invention includes a high drug load solid oral pharmaceutical composition of dexamethasone comprising from about 26% to about 50% by weight of dexamethasone, preferably from about 27.5% to about 35% by weight of dexamethasone based on the total weight of the composition, wherein the composition is free of lactose.

In another embodiment, the present invention includes a high drug load solid oral pharmaceutical composition of dexamethasone comprising from about 26% to about 50% by weight of dexamethasone, preferably from about 27.5% to about 35% by weight of dexamethasone based on the total weight of the composition, wherein the composition is substantially free of other undesirable polymorphic forms.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, disintegrant, glidant, surfactant, and lubricant, wherein the said composition is substantially free of a binder.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, disintegrant, glidant, surfactant, and lubricant, wherein the said composition is completely free of a binder.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, binder, disintegrant, and lubricant, wherein the binder is present in an intra-granular part of the composition in dispersion form (granulation liquid/binder solution) and the intra-granular part of the composition is free of any dry binder.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, binder, disintegrant, glidant, surfactant, and lubricant, wherein the binder is present only in dry form in the intra-granular part of the composition.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, binder, disintegrant, glidant, surfactant, and lubricant, wherein the composition is free of any starch excipient as a dry binder in the intra-granular part of the composition.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, binder, glidant, surfactant, and lubricant, wherein the composition is substantially free of disintegrant.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, binder, glidant, surfactant, and lubricant, wherein the composition is completely free of disintegrant.

In another embodiment of the invention, there is provided a high drug load solid oral pharmaceutical composition comprising dexamethasone or its pharmaceutically acceptable salts or solvates thereof and at least one or more pharmaceutically acceptable excipients including a diluent, binder, disintegrant, glidant, surfactant, and a lubricant, wherein the disintegrant is present either in the intra-granular or in the extra-granular part of the composition. In another embodiment, where the disintegrant is present in the intra-granular part of the composition then the extra-granular part of the composition is free of disintegrant. In still another embodiment, where the disintegrant is present in the extra-granular part of the composition then the intra-granular part of the composition is free of disintegrant.

In another embodiment of the invention, there is provided a high drug load solid oral pharmaceutical composition comprising granules of dexamethasone or its pharmaceutically acceptable salts or solvates thereof and at least one or more pharmaceutically acceptable excipients including a diluent, binder, disintegrant, glidant, surfactant, and a lubricant, wherein the disintegrant is essentially present in both intra-granular as well as extra-granular portion of the composition in equal proportion.

In another embodiment of the invention, there is provided a high drug load solid oral pharmaceutical composition comprising: i) about 26% to about 50% by weight of dexamethasone or its pharmaceutically acceptable salts or solvates thereof; ii) from 0 to about 70% by weight of one or more diluents; iii) from 0 to about 30% by weight of one or more binders; iv) from 0 to about 20% by weight of one or more disintegrants, and v) from 0.1% to about 5% by weight of one or more lubricants. In another embodiment of the invention, the composition is free of any binder.

In another embodiment of the invention, there is provided a high drug load solid oral pharmaceutical composition comprising: i) about 26% to about 50% by weight of dexamethasone or its pharmaceutically acceptable salts or solvates thereof; ii) from 0 to about 70% by weight of one or more diluents; iii) from 0 to about 30% by weight of one or more binders; iv) from 0 to about 20% by weight of one or more disintegrants; v) from 0 to about 10% by weight of one or more surfactants; vi) from 0 to about 10% by weight of one or more glidants, and vii) from about 0.1% to about 5% by weight of one or more lubricants.

In another embodiment of the invention, there is provided a high drug load solid oral pharmaceutical composition comprising: i) about 26% to about 50% by weight of dexamethasone or its pharmaceutically acceptable salts or solvates thereof; ii) from 0 to about 70% by weight of one or more diluents; iii) from 0 to about 30% by weight of one or more binders; iv) from 0 to about 20% by weight of one or more disintegrants, and v) from about 0.1% to about 5% by weight of one or more lubricants, wherein the composition is free of any starch excipient.

In another embodiment of the invention, there is provided a high drug load solid oral pharmaceutical composition comprising: i) about 26% to about 35% by weight of dexamethasone or its pharmaceutically acceptable salts or solvates thereof; ii) from 0 to about 70% by weight of one or more diluents; iii) from 0 to about 30% by weight of one or more binders; iv) from 0 to about 20% by weight of one or more disintegrants, and v) from about 0.1% to about 5% by weight of one or more lubricants.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from 0 to about 10% by weight of one or more binders; iv) from 0 to about 10% by weight of one or more disintegrants; v) from 0 to about 5% by weight of one or more glidant; vi) from 0 to about 5% by weight of one or more surfactants and vii) from about 0.1% to about 5% by weight of one or more lubricants.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more binders; iv) from about 0.01% to about 10% by weight of one or more disintegrants; v) from about 0.01% to about 5% by weight of one or more glidant, and vi) from about 0.1% to about 5% by weight of one or more lubricants.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more binders; iv) from about 0.01% to about 10% by weight of one or more disintegrants; v) from about 0.01% to about 5% by weight of one or more surfactants, and vi) from about 0.1% to about 5% by weight of one or more lubricants.

In another embodiment of the invention, there is provided a high drug load solid oral pharmaceutical composition comprising: i) about 26% to about 35% by weight of dexamethasone or its pharmaceutically acceptable salts or solvates thereof; ii) from about 30% to about 70% by weight of one or more diluents; iii) from about 0.01% to about 10% by weight of one or more binders; iv) from about 0.01% to about 10% by weight of one or more disintegrants; v) from about 0.01% to about 20% by weight of one or more surfactants, and vi) from about 0.1% to about 5% by weight of one or more lubricants.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more disintegrants; and iv) from about 0.1% to about 5% by weight of one or more lubricants, wherein the composition is free of a binder.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more disintegrants; iv) from about 0.01% to about 5% by weight of one or more glidants; v) from about 0.1% to about 5% by weight of one or more surfactants; and vi) from about 0.1% to about 5% by weight of one or more lubricants, wherein the composition is free of a binder.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more disintegrants; iv) from about 0.01% to about 5% by weight of one or more glidant; and v) from about 0.1% to about 5% by weight of one or more lubricants, wherein the composition is free of a binder.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more disintegrants; iv) from about 0.1% to about 5% by weight of one or more surfactants; and v) from about 0.1% to about 5% by weight of one or more lubricants, wherein the composition is free of a binder.

In another embodiment of the invention, there is provided a high drug load solid oral pharmaceutical composition comprising: i) about 27.5% to about 35% by weight of dexamethasone or its pharmaceutically acceptable salts or solvates thereof; ii) from about 50% to about 70% by weight of one or more diluents; iii) from about 0.01% to about 10% by weight of one or more disintegrants, and iv) from about 0.1% to about 5% by weight of one or more lubricants, wherein the composition is free of a binder.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more binders; and iv) from about 0.1% to about 5% by weight of one or more lubricants, wherein the composition is free of disintegrant.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more binders; iv) from about 0.01% to about 5% by weight of one or more glidants; v) from about 0.01% to about 5% by weight of one or more surfactants; and vi) from about 0.1% to about 5% by weight of one or more lubricants, wherein the composition is free of disintegrant.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more binders; iv) from about 0.01% to about 5% by weight of one or more surfactants; and v) from about 0.1% to about 5% by weight of one or more lubricants, wherein the composition is free of disintegrant.

In another embodiment of the invention, there is provided a high drug load solid oral pharmaceutical composition comprising: i) about 26% to about 35% by weight of dexamethasone or its pharmaceutically acceptable salts or solvates thereof; ii) from about 50% to about 70% by weight of one or more diluents; iii) from about 0.01% to about 10% by weight of one or more binders; iv) from about 0.01% to about 5% by weight of one or more glidants; and v) from about 0.1% to about 5% by weight of one or more lubricants, wherein the composition is free of disintegrant.

In another embodiment of the invention, there is provided a high drug load solid oral pharmaceutical composition comprising: i) about 27.5% to about 50% by weight of dexamethasone or its pharmaceutically acceptable salts or solvates thereof; ii) from about 30% to about 60% by weight of one or more diluents; iii) from about 0.01% to about 20% by weight of one or more binders; iv) from about 0.01% to about 20% by weight of one or more disintegrants, and v) from about 0.1% to about 5% by weight of one or more lubricants.

In another embodiment of the invention, there is provided a high drug load solid oral pharmaceutical composition comprising: i) about 27.5% to about 50% by weight of dexamethasone or its pharmaceutically acceptable salts or solvates thereof; ii) from about 30% to about 60% by weight of one or more diluents; iii) from about 0.01% to about 20% by weight of one or more binders; iv) from about 0.01% to about 20% by weight of one or more disintegrants, and v) from about 0.1% to about 5% by weight of one or more lubricants, wherein the composition is free of any starch excipient.

In another embodiment of the invention, there is provided a high drug load solid oral pharmaceutical composition comprising: i) about 27.5% to about 35% by weight of dexamethasone or its pharmaceutically acceptable salts or solvates thereof; ii) from about 50% to about 70% by weight of one or more diluents; iii) from about 0.01% to about 10% by weight of one or more binders; iv) from about 0.01% to about 10% by weight of one or more disintegrants; v) from about 0.01% to about 5% by weight of one or more glidant; vi) from about 0.01% to about 5% by weight of one or more surfactants and vii) from about 0.1% to about 5% by weight of one or more lubricants.

In another embodiment of the invention, one or more diluents are selected from the group consisting of lactose, mannitol, microcrystalline cellulose, silicified microcrystalline cellulose, starch, pregelatinized starch, and dicalcium phosphate.

In another embodiment of the invention, one or more binders are selected from a group consisting of hydroxypropyl methylcellulose, povidone, starch, and microcrystalline cellulose.

In another embodiment of the invention, one or more disintegrants are selected from the group consisting of pregelatinized starch, sodium starch glycolate, crospovidone, croscarmellose sodium, and microcrystalline cellulose.

In another embodiment of the invention, one or more glidants are selected from the group consisting of talc, magnesium silicate, colloidal silicon dioxide, amorphous silicon dioxide, and calcium silicate.

In another embodiment of the invention, one or more surfactants are selected from the group consisting of sodium lauryl sulphate, polysorbate, and poloxamers, and other commercially available surfactants such as SEPITRAP® 80 or SEPITRAP® 4000.

In another embodiment of the invention, one or more lubricants are selected from the group consisting of sodium stearyl fumarate, stearic acid, zinc stearate, magnesium stearate, calcium stearate, and hydrogenated vegetable oil.

In another embodiment of the invention, there is provided a high drug load solid oral pharmaceutical composition comprising dexamethasone or its pharmaceutically acceptable salts or solvates thereof prepared by wet granulation, extrusion-spheronization, dry granulation, dry blending, dry mixing, or direct compression process.

In another embodiment of the invention, there is provided a high drug load solid oral pharmaceutical composition comprising dexamethasone or its pharmaceutically acceptable salts or solvates thereof, the composition prepared in a process comprising the steps of: a) sifting the accurately weighed quantities of active agent and one or more pharmaceutically acceptable excipient(s) separately through a suitable sieve; b) mixing of sifted active agent and excipient(s) using a dry mixer; c) lubricating the sifted blend of step b), and d) compressing the lubricated blend into tablets.

In another embodiment of the invention, there is provided a high drug load solid oral pharmaceutical composition comprising dexamethasone or its pharmaceutically acceptable salts or solvates thereof, the composition prepared in a process comprising the steps of: a) sifting the accurately weighed quantities of active agent and one or more pharmaceutically acceptable excipient(s) through a suitable sieve followed by mixing; b) granulating the mixture of step a) with a binder solution (granulation in an aqueous or non-aqueous solvent); c) drying the granulated mass, optionally milling of the dried granules and mixed the sifted granules; d) lubricating the sifted blend of step c); and e) compressing the lubricated granules into tablets.

In another embodiment of the invention, there is provided a high drug load solid oral pharmaceutical composition comprising dexamethasone or its pharmaceutically acceptable salts or solvates thereof, the composition prepared in a process comprising the steps of: a) blending a mixture of dexamethasone and at least one pharmaceutically acceptable excipient including a binder and/or a disintegrant; b) compacting the blended material using roller compactor; c) optionally milling the compacted material; d) blend from step b) or milled material from step c) was lubricated with a suitable lubricant; and e) lubricating blend or granules from d) and was compressing into tablets with suitable tooling.

In another embodiment of the invention, there is provided a high drug load solid oral pharmaceutical composition comprising dexamethasone or its pharmaceutically acceptable salts or solvates thereof, the composition prepared in a process comprising the steps of: a) sifting the accurately weighed quantities of active agent and one or more pharmaceutically acceptable excipient(s) through a suitable sieve followed by mixing; b) granulating the mixture of step a) with a binder solution (granulation in an aqueous or non-aqueous solvent); c) conveying the obtained wet mass obtained from step b) through an extruder; d) spheronization of the rods obtained from step c); e) drying the granules, optionally milling of the dried granules and mixed the sifted granules; f) lubricating the sifted blend of step e); and g) compressing the lubricated granules into tablets.

In another embodiment of the invention, there is provided a high drug load solid oral pharmaceutical composition comprising dexamethasone or its pharmaceutically acceptable salts or solvates thereof and one or more pharmaceutically acceptable excipients, wherein dexamethasone has a particle size distribution D₉₀ less than about 200 μm, D₅₀ less than about 100 μm and D₁₀ less than about 50 μm. Preferably, D₉₀ less than about 100 μm, D₅₀ less than about 50 μm, and D₁₀ less than about 20 μm.

In another embodiment of the invention, there is provided a high drug load solid oral pharmaceutical composition comprising dexamethasone or its pharmaceutically acceptable salts or solvates thereof and one or more pharmaceutically acceptable excipients, which exhibited desired pharmaceutical technical attributes such as disintegration, dissolution, improved flow characteristics such as bulk density, tapped density, Hausner ratio, compressibility index, stability and bioequivalence that is comparable to the commercially available counterpart (HEMADY® Tablets).

In another embodiment of the invention, there is provided a high drug load solid oral pharmaceutical composition of the present invention in the manufacture of a medicament for treating multiple myeloma, allergy states, dermatological diseases, endocrine disorders, gastrointestinal diseases, hematological diseases, neoplastic diseases, nervous system disorders, ophthalmic diseases, renal diseases, respiratory diseases, rheumatic diseases, trichinosis, and tuberculous meningitis.

Various useful fillers or diluents include, but are not limited to microcrystalline cellulose (“MCC”), sodium alginate, silicified MCC (e.g., PROSOLV™), microfine cellulose, lactitol, cellulose acetate, kaolin, glucose, lactose, maltose, fructose, sucrose, trehalose, starch, pregelatinized starch, mannitol, xylitol, maltitol, sorbitol, dextrates, dextrin, maltodextrin, compressible sugar, confectioner's sugar, dextrose, polydextrose, simethicone, calcium carbonate, calcium sulfate, calcium phosphate, dibasic calcium phosphate dihydrate, tribasic calcium phosphate, magnesium carbonate, magnesium oxide, isomalt, and mixtures thereof. The amount of diluent according to the present invention ranges from 0 to about 90% by weight of the composition. Preferably, the amount of diluent according to the present invention ranges from 0 to about 70% by weight of the composition. In an embodiment, the diluent according to the present invention is present in an amount of about 90% or less, 80% or less, e.g. 70% or less, 60% or less, 50% or less, 40% or less, 30% or less, 20% or less. More preferably, diluent according to the present invention is present in an amount of about 30% to about 80% by weight of the composition.

Various useful binders include, but are not limited to acacia, guar gum, xanthan gum, alginic acid, sodium alginate, dextrin, carbomer, maltodextrin, methylcellulose, ethylcellulose, hydroxyethyl cellulose, hydroxypropyl cellulose (HPC) (e.g., KLUCEL®), hydroxypropyl methylcellulose (HPMC) (e.g., METHOCEL®), hydroxyethyl methyl cellulose, carboxymethyl cellulose sodium, cottonseed oil, povidone (various grades of KOLLIDON®, PLASDONE®), ceratonia, dextrose, polydextrose, starch, gelatin, pregelatinized starch, hydrogenated vegetable oil type I, maltodextrin, microcrystalline cellulose, polyethylene oxide, polymethacrylates and mixtures thereof. Binder can be present in powder form or as a dispersion or mixture of both in intra and/or extra granular part of the composition. The amount of binder according to the present invention ranges from 0 to about 50% by weight of the composition. Preferably, the amount of binder according to the present invention ranges from 0 to about 30% by weight of the composition. In an embodiment, the binder according to the present invention is present in an amount of about 50% or less, 40% or less, e.g. 30% or less, 20% or less, 10% or less, 5% or less. More preferably, binder according to the present invention is present in an amount of about 0.01% to about 30% by weight of the composition.

Various useful disintegrants and/or super-disintegrants include, but are not limited to croscarmellose sodium, carboxymethyl cellulose sodium, carboxymethyl cellulose calcium, povidone, crospovidone, polacriilin potassium, sodium starch glycolate, alginic acid, sodium alginate, calcium phosphate tribasic, docusate sodium, guar gum, low substituted hydroxypropyl cellulose (L-HPC), magnesium aluminum silicate, methylcellulose, microcrystalline cellulose, silicified microcrystalline cellulose, starch, pregelatinized starch and/or combinations thereof. The disintegrant according to the present invention can be used in intra-granular part or extra-granular or in both parts of the composition in any proportion. The amount of disintegrant according to the present invention ranges from 0 to about 40% by weight of the composition. Preferably, the amount of disintegrant according to the present invention ranges from 0 to about 20% by weight of the composition. In an embodiment, the disintegrant according to the present invention is present in an amount of about 40% or less, 30% or less, e.g. 20% or less, 10% or less. More preferably, disintegrant according to the present invention is present in an amount of about 0.01% to about 20% by weight of the composition.

Pharmaceutically acceptable lubricants include stearic acid, zinc stearate, sucrose stearate, sodium benzoate, hydrogenated vegetable oil, calcium stearate, adipic acid, glyceryl palmitostearate, glycerine monostearate, medium-chain triglycerides, glyceryl behenate, sodium lauryl sulphate, sodium stearyl fumarate, magnesium lauryl sulphate, magnesium stearate, polyethylene glycol. The amount of lubricant according to the present invention ranges from 0 to about 20% by weight of the composition. Preferably, the amount of lubricant according to the present invention ranges from about 0.1% to about 5% by weight of the composition. In an embodiment, the lubricant according to the present invention is present in an amount of about 20% or less, e.g. 10% or less, 5% or less. More preferably, lubricant according to the present invention is present in an amount of about 0.01% to about 5% by weight of the composition.

Suitable surfactants according to the present invention are selected from ionic or non-ionic surfactants. Various useful surfactants include, but not limited to, sodium lauryl sulphate, polysorbates (e.g. polysorbate 20, polysorbate 40, polysorbate 60, and polysorbate 80), cetrimide, cetyl alcohol, stearyl alcohol, cetyl stearyl alcohol, cholesterol, polyethylene glycols, polyglycerin fatty acid esters such as decaglyceryl monolaurate and decaglyceryl monomyristate, sorbitan fatty acid esters such as sorbitan monostearate, polyoxyethylene sorbitan fatty acid esters such as polyoxyethylene sorbitan monooleate, polyoxyethylene alkyl ether such as polyoxyethylene lauryl ether, polyoxyethylene castor oil, polyoxyethylene polyoxypropylene block copolymers, and combinations thereof. Commercially available surfactants such as SEPITRAP® 80 or SEPITRAP® 4000 may also be used. SEPITRAP® 80 comprises from about 35% w/w to about 55% w/w of polysorbate 80 and from about 45% w/w to about 65% w/w of magnesium aluminum silicate. The pharmaceutical composition of the present invention may comprise SEPITRAP® 80 in an amount of from about 0.01% w/w to about 20% w/w of the total composition. The amount of surfactant according to the present invention ranges from 0 to about 30% by weight of the composition. In an embodiment, the surfactant according to the present invention is present in an amount of about 30% or less, e.g. 20% or less, 10% or less, 5% or less, or 2% or less. More preferably, surfactant according to the present invention is present in an amount of about 0.01% to about 5% by weight of the composition.

Suitable glidants include, but are not limited to, calcium silicate, magnesium silicate, magnesium trisilicate, stearic acid and its derivatives or esters like magnesium stearate, calcium stearate, and sodium stearate and the corresponding esters such as sodium stearyl fumarate, talc, and colloidal silicon dioxide, tribasic calcium phosphate, starch or mixtures thereof. The amount of glidant according to the present invention ranges from 0 to about 20% by weight of the composition. In an embodiment, the glidant according to the present invention is present in an amount of about 20% or less, e.g. 10% or less, 5% or less, 3% or less. More preferably, glidant according to the present invention is present in an amount of about 0.01% to about 5% by weight of the composition.

Other carrier materials (such as solid carrier, anti-adherents, solubilizer, stabilizer, colorants, flavors, sweeteners, and preservatives) that are known in the pharmaceutical arts may be included in the composition of the present invention.

The high drug load solid oral tablet dosage form prepared by the above process can be subjected to in vitro dissolution evaluation according to Test 711 “Dissolution” in the United States Pharmacopoeia 37, United States Pharmacopoeial Convention, Inc., Rockville, Md., 2014 (“USP”) to determine the rate at which the active substance is released from the dosage form, and the content of the active substance can be determined in solution by high-performance liquid chromatography. When comparing the test and reference products, dissolution profiles should be compared using a similarity factor (f2). The similarity factor is a logarithmic reciprocal square root transformation of the sum of squared error and is a measurement of the similarity in the percent (%) of dissolution between the two curves. Two dissolution profiles are considered similar when the f2 value is equal to or greater than 50.

f2=50·log{[1+(1/n)Σt=1n(Rt−Tt)2]−0.5·100}

Embodiments of the present invention also relate to high drug load solid oral pharmaceutical composition comprising dexamethasone or its pharmaceutically acceptable salts or solvates thereof and one or more pharmaceutically acceptable excipients, wherein the composition exhibits more than 80% of drug release within 30 minutes in 900 ml of dilute Hydrochloric acid, using USP II apparatus (Paddle) at a temperature of 37±0.5° C. and a rotation speed of 50 revolutions per minute. In another embodiment, the composition exhibits more than 70% of drug release within 45 minutes in 900 ml of dilute Hydrochloric acid (1 in 100), using USP I apparatus (Basket) at a temperature of 37±0.5° C. and a rotation speed of 50 revolutions per minute. In another embodiment, the composition exhibits more than 70% of drug release within 45 minutes in 500 ml of 0.1N Hydrochloric acid, using USP II apparatus (Paddle) at a temperature of 37±0.5° C. and a rotation speed of 50 revolutions per minute.

In another embodiment, high drug load solid oral pharmaceutical compositions of the present invention particularly tablet dosage form may be packaged in HDPE bottles or blister packs. HDPE bottles may optionally contain desiccants.

In accordance with still another embodiment of the present invention, there is provided use of high drug load solid oral pharmaceutical compositions of the present invention in the manufacture of a medicament for treating multiple myeloma in combination with at least one anti-cancer drug, allergy states, dermatological diseases, endocrine disorders, gastrointestinal diseases, hematological diseases, neoplastic diseases, nervous system disorders, ophthalmic diseases, renal diseases, respiratory diseases, rheumatic diseases, trichinosis, and tuberculous meningitis.

The invention is further defined by reference to the following examples describing in detail method for the preparation and testing of dexamethasone pharmaceutical composition. It will be apparent to those skilled in the art that many modifications, both to materials and methods, may be practiced without departing from the scope of the invention. The following examples are set out to illustrate the invention and do not limit the scope of the present invention.

### Example 1

Dexamethasone tablets were prepared by using the quantitative formula as given in Table 1: (Quantity/Tablet (% w/w)).

TABLE 1
Examples 1-4

| S. No. | Ingredient | Function | 1 | 2 | 3 | 4 |
|---|---|---|---|---|---|---|
| 1. | Dexamethasone | Active | 26-50 | 26-35 | 27.5-50 | 27.5-35 |
| | | Ingredient | | | | |
| 2. | Lactose/ | Diluent | 0-70 | 30-60 | 30-60 | 30-60 |
| | Microcrystalline cellulose/Mannitol/ | | | | | |
| | Dicalcium phosphate/Microcrystalline | | | | | |
| | cellulose | | | | | |
| 3. | Povidone/Hypromellose/Starch/ | Binder | 0-30 | 0-20 | 0-20 | 0.01-20 |
| | Microcrystalline cellulose | | | | | |
| 4. | Sodium starch glycolate/ | Disintegrant | 0-20 | 0-10 | 0-20 | 0.01-20 |
| | Croscarmellose sodium/Crospovidone/ | | | | | |
| | Pre-Gelatinized starch/ | | | | | |
| | Microcrystalline cellulose | | | | | |
| 5. | Sodium Lauryl Sulphate | Surfactant | 0-2 | 0-2 | 0-2 | — |
| 6. | Magnesium Stearate | Lubricant | 0.1-5 | 0.1-3 | 0.1-2 | 0.1-2 |

Preparation Method I: i) Dexamethasone, diluent, and other optional excipients such as disintegrant, binder, and surfactant were sifted through a suitable sieve. ii) The sifted blend of step i) was mixed in a suitable blender for a suitable time and compacted using a roller compactor. iii) The compacts from step ii) were milled using a suitable sieve. iv) The blend from step iii) was mixed with an optional disintegrant and further lubricated with lubricant. v) The blend of step iv) was compressed into tablets using suitable punches.
Preparation Method II: i) Dexamethasone, diluent, and other optional excipients such as disintegrant, binder, and surfactant were sifted through a suitable sieve. ii) The sifted blend of step i) was mixed for a suitable time. iii) Binder was dissolved in purified water to prepare a binder solution. iv) Step ii) blend was granulated using the binder solution of step iii). v) The granules of step iv) were dried and optionally sifted and/or milled through a suitable sieve. vi) The granules of step v) were mixed with an optional disintegrant and then lubricant was sifted through a suitable sieve and added to granules and mixed for a suitable time. vii) The blend of step vi) was compressed into tablets using suitable punches.

TABLE 2
Examples 5-19: (Quantity/Tablet (% w/w))

| S. No. | Ingredient | Function | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|
| 1 | Dexamethasone | Active | 26-35 | 27.5 | 27.5 | 28.6 | 25.9 | 28.6 | 28.6 | 28.6 | 28.6 | 28.6 | 28.6 | 28.6 | 27.6 | 35.0 | 35.0 |
| | | Ingredient | | | | | | | | | | | | | | | |
| 2 | Silicified MCC | Diluent | — | — | — | 66.5 | 60.3 | — | — | — | — | — | — | | — | — | — |
| 3 | MCC | Diluent | — | 55.4 | — | — | — | — | — | — | — | — | — | — | — | 61.7 | — |
| 4 | Lactose | Diluent | 50-70 | — | 32.5 | — | — | 32.9 | 30.9 | 36.4 | 65.9 | 65.9 | 60.0 | 32.4 | 64.1 | — | 61.7 |
| 5 | Pregelatinized | Diluent | — | — | 11.7 | — | — | 25.0 | 25.0 | 25.0 | — | — | — | 25.0 | — | — | — |
| | starch | | | | | | | | | | | | | | | | |
| 6 | Povidone | Binder | 0-10 | 3.6 | 8.3 | — | 2.7 | 3.6 | 3.6 | — | — | 3.0 | 3.0 | — | 3.0 | 3.0 | — |
| 7 | Sodium starch | Disintegrant | 0-10 | — | 4.0 | 3.0 | — | 8.6 | 8.6 | 8.6 | — | — | — | 8.6 | — | — | — |
| | glycolate | | | | | | | | | | | | | | | | |
| 8 | Crospovidone | Disintegrant | — | 8.6 | — | — | — | — | — | — | 3.0 | — | — | — | 2.9 | — | 3.0 |
| 9 | Talc | Glidant | 0-5 | — | — | 1.5 | 4.5 | — | — | — | 2.1 | 2.0 | 2.0 | — | 2.0 | — | — |
| 10 | Colloidal | Glidant | — | 1.5 | — | — | 2.0 | — | — | — | — | — | — | — | — | — | — |
| | Silicon dioxide | | | | | | | | | | | | | | | | |
| 11 | Sodium Lauryl | Surfactant | 0-5 | 2.0 | — | — | — | — | 2.0 | — | — | — | 5.0 | 4.0 | — | — | — |
| | Sulphate | | | | | | | | | | | | | | | | |
| 12 | Sepitrap-80 | Surfactant | 0-20 | — | 15.3 | — | — | — | — | — | — | — | — | — | — | — | — |
| 13 | Magnesium | Lubricant | 0.1-5 | 1.4 | 0.7 | 0.4 | 4.5 | 1.4 | 1.4 | 1.4 | 0.4 | 0.5 | 1.4 | 1.4 | 0.4 | 0.4 | 0.4 |
| | Stearate | | | | | | | | | | | | | | | | |

Preferred method of manufacture: wet granulation, dry granulation, direct compression, and extrusion-spheronization.
Preparation Method I: i) Dexamethasone, diluent, and other optional excipients such as disintegrant, binder, and surfactant were sifted through a suitable sieve. ii) The sifted blend of step i) was mixed for a suitable time. iii) Binder and other optional excipients were added in purified water or any other suitable non-aqueous solvent to prepare a binder solution/dispersion. iv) Step ii) blend was granulated using the binder solution/dispersion of step iii). v) The granules of step iv) were dried and optionally sifted and/or milled through a suitable sieve. vi) The granules of step v) were mixed with an optional disintegrant, glidant, and then lubricant was sifted through a suitable sieve and added to granules, and mixed for a suitable time. vii) The blend of step vi) was compressed into tablets using suitable punches.
Preparation Method II: i) Dry mixing of dexamethasone with other optional excipients such as diluent, surfactant, the first portion of disintegrant, and binder; ii) Binder was dissolved in purified water to prepare a binder solution/dispersion. iii) Blend was granulated using the binder solution/dispersion of step ii); iv) The granules of step iii) were dried and optionally sifted and/or milled through a suitable sieve; v) The remaining second portion of disintegrant was added followed by the addition of lubricant and mixed for a suitable time. vi) The blend of step v) was compressed into tablets using suitable punches.
Preparation Method III: i) The accurately weighed quantities of active agent and other optional excipients such as disintegrant, binder, and surfactant were sifted through a suitable sieve; ii) The sifted active agent and excipients were mixed using a suitable blender; iii) The sifted blend of step ii) was mixed with glidant and then lubricated, and iv) The lubricated blend was compressed into tablets.
Results: Dissolution: The dissolution profile of tablet compositions prepared using quantitative compositions, was measured in 500 ml of 0.1N Hydrochloric acid, using USP II apparatus (Paddle) at a temperature of 37±0.5° C. and a rotation speed of 50 revolutions per minute. The quantitative composition exhibited more than 70% of drug release within 45 minutes, accordingly dissolution profiles were found acceptable.
While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope, and spirit of this invention.

What is claimed:

1. A high drug load solid oral pharmaceutical composition comprising:
i) about 26% to about 50% by weight of dexamethasone or its pharmaceutically acceptable salts or solvates thereof;
ii) from 0 to about 70% by weight of one or more diluents;
iii) from 0 to about 30% by weight of one or more binders;
iv) from 0 to about 20% by weight of one or more disintegrants; and
v) from 0.1% to about 5% by weight of one or more lubricants.

2. The high drug load solid oral pharmaceutical composition according to claim 1, further comprises one or more pharmaceutically acceptable excipients selected from glidant and surfactant.

3. The high drug load solid oral pharmaceutical composition according to claim 1, wherein the composition is substantially free of any starch excipient.

4. The high drug load solid oral pharmaceutical composition according to claim 1, wherein the composition is free of any dry binder in an intra-granular part of the composition.

5. The high drug load solid oral pharmaceutical composition according to claim 1, wherein the composition is free of any binder.

6. The high drug load solid oral pharmaceutical composition according to claim 1, wherein the composition comprises dexamethasone or its pharmaceutically acceptable salts or solvates thereof in an amount of about 27.5% or more.

7. The high drug load solid oral pharmaceutical composition according to claim 1, wherein the diluent comprises one or more of lactose, mannitol, microcrystalline cellulose, silicified microcrystalline cellulose, starch, pregelatinized starch, and dicalcium phosphate.

8. The high drug load solid oral pharmaceutical composition according to claim 1, wherein the binder comprises one or more of hydroxypropyl methylcellulose, povidone, starch, and microcrystalline cellulose.

9. The high drug load solid oral pharmaceutical composition according to claim 1, wherein the disintegrant comprises one or more of pregelatinized starch, sodium starch glycolate, crospovidone, croscarmellose sodium, and microcrystalline cellulose.

10. The high drug load solid oral pharmaceutical composition according to claim 2, wherein the glidant comprises one or more of talc, magnesium silicate, colloidal silicon dioxide, amorphous silicon dioxide, and calcium silicate.

11. The high drug load solid oral pharmaceutical composition according to claim 2, wherein the surfactant comprises one or more of sodium lauryl sulphate, polysorbate, and poloxamers.

12. The high drug load solid oral pharmaceutical composition according to claim 1, prepared by wet or dry granulation process.

13. The high drug load solid oral pharmaceutical composition according to claim 1, prepared by the direct compression process.

14. The high drug load solid oral pharmaceutical composition according to claim 1, wherein the composition is in the form of a tablet.

15. A high drug load immediate-release tablet composition comprising:
i) about 26% to about 35% by weight of dexamethasone or its pharmaceutically acceptable salts or solvates thereof;
ii) from about 50% to about 70% by weight of one or more diluents;
iii) from about 0.01% to about 10% by weight of one or more disintegrants; and
iv) from about 0.1% to about 5% by weight of one or more lubricants;
wherein the composition is free of a binder.

16. A high drug load immediate-release tablet composition comprising:
i) about 26% to about 35% by weight of dexamethasone or its pharmaceutically acceptable salts or solvates thereof;
ii) from about 50% to about 70% by weight of one or more diluents;
iii) from about 0.01% to about 10% by weight of one or more binders; and
iv) from about 0.1% to about 5% by weight of one or more lubricants;
wherein the composition is free of a disintegrant.

17. The high drug load immediate-release tablet composition according to claim 15, wherein the composition comprises 20 mg of dexamethasone.

18. The high drug load immediate-release tablet composition according to claim 15, wherein the composition further comprises from about 0.01% to about 5% by weight of one or more glidants.

19. The high drug load immediate-release tablet composition according to claim 15, wherein the composition further comprises from about 0.01% to about 5% by weight of one or more surfactants.

20. The high drug load immediate-release tablet composition according to claim 15, wherein the composition releases more than 70% of drug within 45 minutes in 500 ml of 0.1N Hydrochloric acid, using USP II apparatus (Paddle) at a temperature of 37±0.5° C. and a rotation speed of 50 revolutions per minute.
